(19) United States
(12) Patent Application Publication
(10) Pub. No.: US 2026/0142030 A1
(43) Pub. Date: May 21, 2026

(54) SYSTEM AND METHOD FOR REAL-TIME AI-BASED MEDICAL RECOMMENDATIONS

(71) Applicants: Bianca Gream, Stockhom (SE); Timothy Boyd, Stockhom (SE)

(72) Inventors: Bianca Gream, Stockhom (SE); Timothy Boyd, Stockhom (SE)

(21) Appl. No.: 18/952,900

(22) Filed: Nov. 19, 2024

(51) Int. Cl. G16H 50/20; G06F 40/205; G06N 3/042; G06V 10/82; G06V 20/40; G08B 25/00; G10L 25/30; G10L 25/66; G16H 10/60; G16H 40/67; G16H 80/00

(52) U.S. Cl. CPC G16H 50/20; G06F 40/205; G06N 3/042; G06V 10/82; G06V 20/46; G08B 25/006; G10L 25/30; G10L 25/66; G16H 10/60; G16H 40/67; G16H 80/00; G06V 2201/03

(57) ABSTRACT

A system for generation of medical recommendation based on patient-related data, including a processor of a medical recommendations server (MRS) node configured to host a machine learning (ML) module and connected to a patient entity node and to at least one medical emergency entity node over a network and a memory on which are stored machine-readable instructions that when executed by the processor, cause the processor to: acquire sensory data from a plurality of biosensors encapsulated into a patient wearable device; acquire patient data from a mobile device of the patient, wherein the patient data may include video data and audio data; parse the sensory data to derive a plurality of key classifying features; extract a plurality of indicators from the patient data based on the plurality of key classifying features; query a local patients'database to retrieve local historical patients'-related data related to previous patients'engagements associated with previous medical recommendations based on the plurality of key classifying features and the plurality of indicators; generate at least one feature vector based on the plurality of key classifying features and the plurality of indicators and the local historical patients'-related data; and provide the feature vector to the ML module coupled to an Artificial Neural Network (ANN); receive a plurality of medical recommendation parameters from a medical recommendation predictive model generated by the ML module using outputs of the ANN based on the feature vector; and generate medical recommendations based on the plurality of the medical recommendation parameters.

### FIELD OF DISCLOSURE

The present disclosure generally relates to automated medical alert applications, and more particularly, to an AI-based automated system and method for real-time interactive recommendations based on patient-response data and patient sensory data.

### BACKGROUND

Globally, seizures impact 1 in 10 (795M) people, 48% (380M) will have recurring seizures. Whilst 1 in 100 (79M) will develop Epilepsy. Tragically 266.2 people die each day (97,178 annually). In the UK this translates to over 3.2M people being predisposed to recurring seizures, of which over 630,000 people have already been diagnosed epileptic, with an annual mortality rate of 1,663 and seizure admission growing by 9.7%. Conversely, seizures rates in EDI and lower socioeconomic groups are 4× higher.

Calculated on a human capital methodology in UK, seizures cost £14.9B (and £9B indirect): 3.8M neurological consults and associated imaging, 6M ambulance calls, 3.8M hospital beds, 8M clinical visits (General Practitioner/Nursing), 2.1B anti-seizure medication doses. Indirect and intangible impacts are broad; notably 68% of adults who suffer seizures develop a mental health condition (£3B), whilst 40% of children who suffer seizures will be diagnosed with a mental health condition (£4B), a further 30% develop learning difficulties (£4.8B) and 65% of parents develop a mental health condition requiring treatment (£460M), which leads to a £37M annual impact to productivity and 602,000 loss of working hours.

However, people with seizures are disadvantaged, within society, the healthcare system, economically and developmentally. Despite 25% of seizures being avoidable (direct NHS saving £2.4B) and 70% of seizure patients are able to live seizure free with the correct treatment. Seizure rates and cost continues to grow. Several factors contribute to these disadvantages such as: significant shortage in health workers specialized in neurological health (7:100,000 ratio v. 18:100,000 ratio OECD standard), 40% of EDI groups cannot access appropriate care or are unable to afford expensive private care solutions; lack of digitizing of health records.

Existing systems attempt to deal with remote medical solutions. For example, U.S. Pat. No. 7,188,151 B2 discloses a system for network-based monitoring of physiological data, enabling remote transmission and viewing of patient data over the Internet. It contains patient-side devices collecting physiological data, provider-side devices receiving the data, and an engine managing data transmission. Key features include real-time streaming of raw and processed physiological data, audio/video/text communication, and support for multiple medical devices using a modular architecture with plug-and-play web drivers. The system offers remote viewing of patient data, alerts for abnormal events, remote device control, and data storage for later analysis through a browser-based interface. Improvements over existing technology include remote monitoring without specialized provider equipment, support for diverse devices in a single system, and easy access to historical data.

US2010/0056877 A1 describes a medical data analysis system that utilizes cloud computing resources for real-time or near real-time processing of patient data. The system contains patient-side sensors or devices that collect medical data, a middleware component that conditions and routes the data, a cloud computing infrastructure for analysis, and user interface devices for healthcare providers. Key features include the ability to sense various medical parameters like vascular flow, ECG, and MRI data; transmit this data securely to the cloud; process it using powerful cloud-based applications that can compare it with historical and demographic data; and return analyzed results quickly to the healthcare provider's device. The middleware plays a crucial role in formatting data appropriately for both the cloud and the end-user devices. The system allows for real-time monitoring during procedures, enabling immediate adjustments based on analysis results. It also supports mobile usage, with portable sensors and user devices that can connect from multiple locations. The patent emphasizes the system's ability to provide quick, comprehensive analysis by leveraging cloud computing power, potentially reducing the need for invasive procedures and improving diagnostic capabilities. Security features like encryption are incorporated to protect sensitive medical data. This invention aims to enhance medical testing efficiency and provide healthcare providers with more detailed, easily accessible information for better patient care.

EP 4385041A1 describes a risk assessment and intervention platform for predicting and reducing negative outcomes in clinical trials. The system uses machine learning models to analyze patient data, intervention protocols, and historical treatment data to generate intervention risk scores. Key features include real-time data processing from wearables and environmental sensors, historical trend analysis, medical insights integration, localization of medical practices, and an AI-based recommendation engine. The platform can automatically assess risk scores and initiate intervention actions when predetermined thresholds are met. It includes components for patient enrollment, tracking, monitoring, and engagement through the trial process. The system supports various data inputs, including biometric data, patient responses, and medication intake information. It can send alerts to medical clinicians or systems when assistance is needed or when a patient is at risk of disengaging from the trial protocol. The platform also incorporates security features and can integrate with existing electronic health record systems. Overall, this invention aims to improve clinical trial efficiency, reduce dropout rates, and enhance the accuracy and timeliness of health insights for patients and healthcare providers.

However, no solution exists to prevent, detect, manage and treat people with seizures in real-time using AI and machine learning solutions.

Accordingly, a system and method for AI-based automated real-time interactive recommendations based on patient-response data and patient sensory data are desired.

### BRIEF OVERVIEW

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

One embodiment of the present disclosure provides a system for generation of medical recommendation based on patient-related data, including a processor of a medical recommendations server (MRS) node configured to host a machine learning (ML) module and connected to a patient entity node and to at least one medical emergency entity node over a network and a memory on which are stored machine-readable instructions that when executed by the processor, cause the processor to: acquire sensory data from a plurality of biosensors encapsulated into a patient wearable device; acquire patient data from a mobile device of the patient, wherein the patient data may include video data and audio data; parse the sensory data to derive a plurality of key classifying features; extract a plurality of indicators from the patient data based on the plurality of key classifying features; query a local patients'database to retrieve local historical patients'-related data related to previous patients'engagements associated with previous medical recommendations based on the plurality of key classifying features and the plurality of indicators; generate at least one feature vector based on the plurality of key classifying features and the plurality of indicators and the local historical patients'-related data; and provide the feature vector to the ML module coupled to an Artificial Neural Network (ANN); receive a plurality of medical recommendation parameters from a medical recommendation predictive model generated by the ML module using outputs of the ANN based on the feature vector; and generate medical recommendations based on the plurality of the medical recommendation parameters.

Another embodiment of the present disclosure provides a method that includes one or more of: acquiring sensory data from a plurality of biosensors encapsulated into a patient wearable device; capturing patient data from a mobile device of the patient, wherein the patient data may include video data and audio data; parsing the sensory data to derive a plurality of key classifying features; extracting a plurality of indicators from the patient data based on the plurality of key classifying features; querying a local patients'database to retrieve local historical patients'-related data related to previous patients'engagements associated with previous medical recommendations based on the plurality of key classifying features and the plurality of indicators; generating at least one feature vector based on the plurality of key classifying features and the plurality of indicators and the local historical patients'-related data; and providing the feature vector to the ML module coupled to an Artificial Neural Network (ANN); receiving a plurality of medical recommendation parameters from a medical recommendation predictive model generated by the ML module using outputs of the ANN based on the feature vector; and generating medical recommendations based on the plurality of the medical recommendation parameters.

Another embodiment of the present disclosure provides a computer-readable medium including instructions for: acquiring sensory data from a plurality of biosensors encapsulated into a patient wearable device; capturing patient data from a mobile device of the patient, wherein the patient data may include video data and audio data; parsing the sensory data to derive a plurality of key classifying features; extracting a plurality of indicators from the patient data based on the plurality of key classifying features; querying a local patients'database to retrieve local historical patients'-related data related to previous patients'engagements associated with previous medical recommendations based on the plurality of key classifying features and the plurality of indicators; generating at least one feature vector based on the plurality of key classifying features and the plurality of indicators and the local historical patients'-related data; and providing the feature vector to the ML module coupled to an Artificial Neural Network (ANN); receiving a plurality of medical recommendation parameters from a medical recommendation predictive model generated by the ML module using outputs of the ANN based on the feature vector; and generating medical recommendations based on the plurality of the medical recommendation parameters.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings may contain representations of various trademarks and copyrights owned by the Applicant. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1A illustrates a network diagram of a system for an AI-based automated real-time interactive recommendations based on predictive analytics of patient-response data and patient sensory data consistent with the present disclosure;

FIG. 1B illustrates a network diagram of a system for an AI-based automated real-time interactive recommendations based on predictive analytics of patient-response data and patient sensory data implemented over a blockchain network consistent with the present disclosure;

FIG. 2 illustrates a network diagram of a system including detailed features of a medical recommendations server (MRS) node consistent with the present disclosure;

FIG. 3A illustrates a flowchart of a method for an AI-based automated real-time interactive recommendations based on predictive analytics of patient-response data and patient sensory data consistent with the present disclosure;

FIG. 3B illustrates a further flowchart of a method for an AI-based automated real-time interactive recommendations based on predictive analytics of patient-response data and patient sensory data consistent with the present disclosure;

FIG. 4 illustrates deployment of a machine learning model for prediction of treatment recommendation parameters using blockchain assets consistent with the present disclosure;

FIG. 5 illustrates a block diagram of a system including a computing device for performing the method of FIGS. 3A and 3B.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such a term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. §112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subject matter disclosed under the header.

The present disclosure includes many aspects and features. Moreover, while many aspects and features relate to, and are described in the context of the predictive analytics of live patient sensory data, embodiments of the present disclosure are not limited to use only in this context.

This innovative system represents a technological advancement in personalized healthcare, integrating multiple data streams with artificial intelligence to provide real-time monitoring, recommendations, and emergency support. At its core, the system uses a sophisticated data integration layer that collects and processes information from a diverse array of sources, including wearable devices, environmental sensors, user inputs, medical literature, and regional health practices. This comprehensive data collection forms the foundation for the system's advanced analytical capabilities.

The system's architecture is built around several key modules, each contributing to its overall functionality. The real-time data processing module continuously analyzes biometric and environmental data, utilizing advanced signal processing algorithms to extract meaningful health indicators. This is complemented by a historical trend analysis module that uses machine learning techniques to identify patterns and anomalies in individual and anonymized group data, enhancing the system's predictive capabilities. A key component of the system is the medical insights integration module, which leverages natural language processing (NLP) and deep learning algorithms to extract and synthesize relevant information from medical journals, guidelines, and the proprietary local and third-party databases. This module continuously updates its knowledge base, ensuring that the system's recommendations are always aligned with the latest medical research and best practices.

The system's point of novelty lies in its AI-based recommendation engine. This engine utilizes a proprietary Risk Assessment Grid (RAG) algorithm that combines supervised and unsupervised learning models with reinforcement learning techniques. The RAG algorithm processes inputs from all other modules to generate highly personalized, real-time health recommendations and emergency response activities. Its ability to dynamically adapt to new data and user feedback sets it apart from traditional rule-based systems.

Another unique feature is the system's open API interface, which allows integration with an unprecedented number of wearable devices. This universal compatibility is achieved through a sophisticated protocol translation layer that standardizes data from diverse sources into a unified format for analysis. This approach overcomes the limitations of proprietary hardware solutions, making the system accessible to a broader user base. The localization module adds another layer of sophistication, using geospatial analysis and regulatory compliance algorithms to adjust recommendations based on region-specific medical practices and guidelines. This ensures that the system's outputs are personalized to the individual and compliant with local healthcare standards and regulations. A standout technical feature is the system's multi-modal communication capability. The system can use advanced natural language understanding and generation models to engage in text-to-text, voice-to-voice, and even image-to-text/voice interactions. This is achieved by integrating state-of-the-art speech recognition, computer vision, and language processing technologies. A secure, blockchain-based data exchange protocol facilitates the system's integration with electronic health record (EHR) platforms. This protocol ensures the integrity and confidentiality of sensitive medical information while enabling seamless communication between the system, users, and healthcare providers.

In summary, the discloses system and method represent an advancement in personalized health management technology. Its novel approach to integrating multiple data sources, leveraging AI for real-time analysis and recommendations, and providing adaptive, localized support addresses many limitations of existing solutions. The system's ability to process vast amounts of data and provide context-aware, personalized recommendations in real-time positions it as a potentially transformative tool in healthcare management and emergency support.

The present disclosure provides a system, method and computer-readable medium for a system and method for an AI-based automated real-time interactive recommendations based on predictive analytics of patient-response (e.g., chat) data and patient sensory data acquired from a wearable device. In one embodiment, the system overcomes the limitations of existing methods of remote medical treatment or diagnosis by employing fine-tuned models to process the user response(s) information, irrespective of data format, style, or data type. By leveraging the capabilities of the pre-trained language models and predictive models, the disclosed approach offers a significant improvement over existing solutions discussed above in the background section.

In one embodiment of the present disclosure, the system provides for an AI and machine learning (ML)-generated medical recommendation parameters. In one embodiment, an automated medical recommendation predictive model may be generated to provide for recommendation parameters associated with the patient being monitored. The automated model may use historical patient-related data collected at the current medical facility location (or site) and at third-party facilities of the same type located within a certain range from the current location or even located globally. The relevant historical patient-related data may include data related to other patients having the same characteristics such as age, gender, race, medical conditions, language of the jurisdiction of the medical facility, locations, etc. The relevant patient-related data may indicate successfully diagnosed and treated conditions based on analytics of the patient-related data.

In one embodiment, to enhance this process, the system may integrate advanced technologies discussed above, such as Artificial Intelligence (AI) and machine-learning (ML) and Blockchain. The AI may be leveraged for several key functions in the manner described herein.

Additionally, the disclosed system may incorporate Blockchain technology to ensure the transparency and immutability of transactions, providing a secure and trustworthy platform. By embedding these advanced technologies, the disclosed automated medical system, advantageously, offers a sophisticated and secure solution.

As discussed above, in one disclosed embodiment, the AI/ML technology may be combined with a blockchain technology for secure use of the patient-related data and treatment-related data. In one embodiment, users (e.g., patients being monitored and treated) may logon into the recommendation system implemented via an AI-based chatbot that may ask questions or provide back additional questions or hints and maintain a responsive conversation. Once the treatment case is closed or completed, a feedback report may be generated and sent to a medical emergency supervisor or other superior in the organization. This feedback my includes a transcript of the treatment and the report may provide notes on what worked well and what could be better and needs improvement, as determined by the medical recommendation predictive model. In one embodiment, a blockchain consensus may need to be implemented prior to provision of the treatment feedback report to the patient who had participated in the emergency treatment session.

In one embodiment, the secure chat channel may be implemented using a Chabot. The treatment-related documents and reports may be stored in a form of uniquely minted NFTs on the private (permissioned) blockchain ledger.

In one embodiment, the ML module may use predictive model(s) that use an artificial neural network (ANN) to generate medical recommendation parameters and update the parameters in live environment. The use of specially trained ANNs provides a number of improvements over traditional methods of analyzing of sensory and chat data received from the patient being monitored, including more accurate prediction of what treatment recommendations may be provided to the patient and/or to the medical provider. The application further provides methods for training the ANN that leads to a more accurate medical recommendation predictive model(s).

In one embodiment, the ANN can be implemented by means of computer-executable instructions, hardware, or a combination of the computer-executable instructions and hardware. In one embodiment, neurons of the ANN may be represented by a register, a microprocessor configured to process input signals. Each neuron produces an output, or activation, based on an activation function that uses the outputs of the previous layer and a set of weights as inputs. Each neuron in a neuron array may be connected to another neuron via a synaptic circuit. A synaptic circuit may include a memory for storing a synaptic weight. A proposed ANN may be implemented as a Deep Neural Network having an input layer, an output layer, and several fully connected hidden layers. The proposed ANN may be particularly useful in medical recommendation updates because the ANN can effectively extract features from the patient sensory data in linear and non-linear relationships. In some embodiments, the proposed ANN may be implemented by an application-specific integrated circuit (ASIC). The ASICs may be specially designed and configured for a specific AI application and provide superior computing capabilities and reduced electricity and computational resources consumption compared to the traditional CPUs.

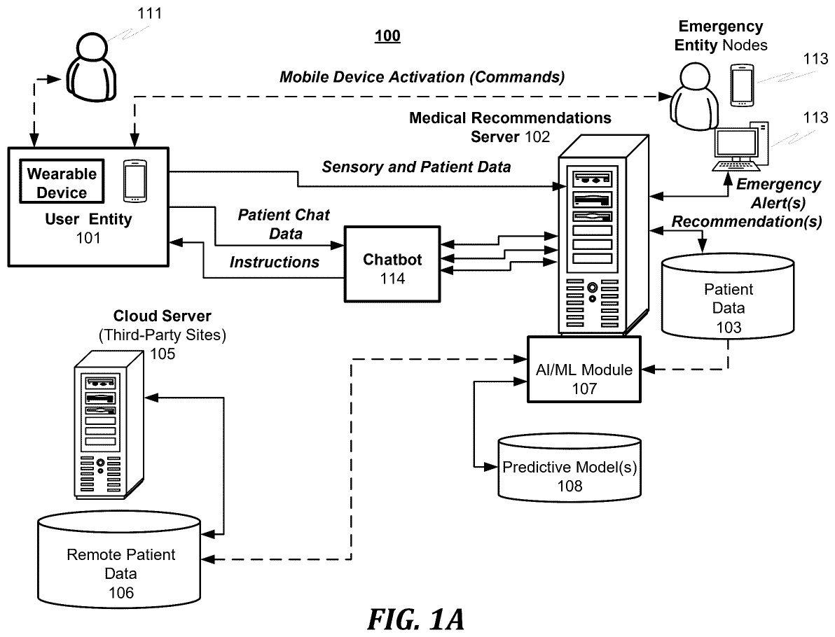

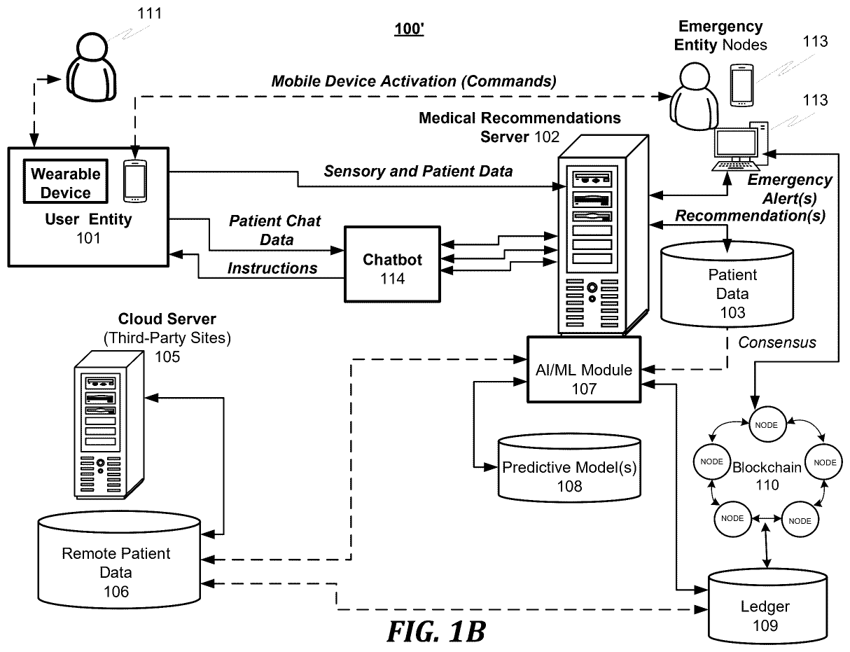

FIG. 1A illustrates a network diagram of a system for an AI-based automated real-time interactive recommendations based on predictive analytics of patient-response data and patient sensory data consistent with the present disclosure.

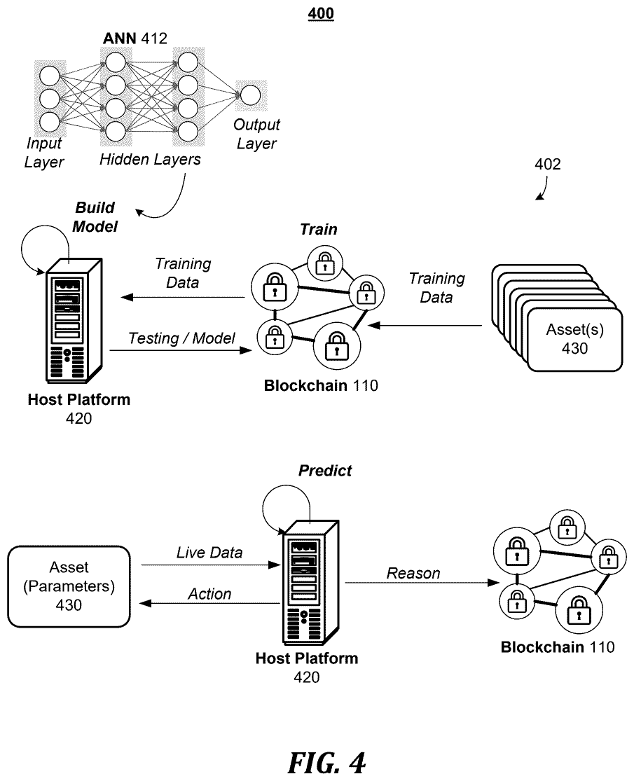

Referring to FIG. 1A, the example network 100 includes the Medical Recommendations Server (MRS) node 102 connected to a cloud server node(s) 105 over a network. The MRS node 102 is configured to host an AI/ML module 107 couple to the ANN (shown in FIG. 4). The MRS node 102 may acquire patient 111 sensory data from a wearable device associated with a user entity node 101.

The MRS node 102 may capture a patient response data from a patient mobile device in a form of patient chat data from the user-entity node 101 associated with the user 111 being monitored via a chatbot conversation agent 114 supported by the AI/ML module 107 of the MRS node 102. The patient chat data may include video, audio or textual data.

The patient chat data may have language indicator metadata representing the language of the patient 111 used during the communication. In one embodiment, the patient response data may be processed by the MRS node 102 using the pre-trained large language models (LLM). The MRS node 102 may derive the language indicator and parse out the user response data based on the language indicator metadata. In other words, the key features of the user response data may be, advantageously, derived from the user response data based on the language of the user response regardless of a form of communication.

In one embodiment, the language indicator may serve as a kind of a linguistic profile associated with the patient 111. The language indicator may guide the AI/ML module 107 in dynamically tailoring the treatment recommendation parameters for the Chabot 114 and ultimately for the patient 111. Depending on the language indicated, the MRS node 102 could engage specialized language models or apply unique natural language processing techniques optimized for that language.

Regarding the global reach of the disclosed system and method, a cultural intelligence layer may be added to the language indicator. The goal of this layer is for the system to not only recognize the language, but also adapt its recommendations and interactions to be culturally sensitive and appropriate for the patient 111. In one embodiment, the disclosed system may employ integrated translation capabilities. This may allow both the patient 111 and the emergency entity nodes 113 associated with the MRS 102 to communicate effortlessly via the Chabot 114 or directly with the patient, no matter where they are in the world or what languages they use. The language indicator metadata may support and/or trigger this feature, making the system truly globally effective.

The MRS node 102 may query a local patient database 103 for the historical local patient data based on the patient data associated with the current patient and sensory data associated with user entity 101 node. The MRS node 102 may acquire relevant remote patient data from a remote database 106 residing on the cloud server 105. The remote patient data in the database 106 may be collected from other third-party medical facilities. The remote patient data may be collected from the patients of the same (or similar) type, age, gender, location, language, race, etc. as the local patient 111 based on a pre-stored patient 111 profile.

The MRS node 102 may generate a feature vector or classifier data based on the patient data and sensory data and the collected heuristics data (i.e., pre-stored local patient data 103 and remote patient data 106). The MRS node 102 may ingest the feature vector/classifier data into an AI/ML module 107. The AI/ML module 107 may generate a medical recommendation predictive model(s) 108 based on the feature vector/classifier data to predict medical recommendation parameters for automatically generating medical recommendations for the patient 111 being monitored. The medical recommendation parameters may be further analyzed by the MRS node 102 prior to generation of the actual recommendation or updating the latest recommendation based on the live sensory data. In one embodiment, the updated recommendation parameters may be used for adjustment of the treatment or emergency alerts. Once the treatment is recorded, the entire or partial treatment data may be analyzed to generate a feedback report by the AI/ML module 107 based on the outputs of the medical recommendation predictive model(s) 108.

FIG. 1B illustrates a network diagram of a system for an AI-based automated real-time interactive recommendations based on predictive analytics of patient-response data and patient sensory data implemented over a blockchain network consistent with the present disclosure.

Referring to FIG. 1B, the example network 100′ includes the Medical Recommendations Server (MRS) node 102 connected to a cloud server node(s) 105 over a network. The MRS node 102 is configured to host an AI/ML module 107 couple to the ANN (shown in FIG. 4). The MRS node 102 may acquire patient 111 sensory data from a wearable device associated with a user entity node 101.

The MRS node 102 may capture a patient response data from a patient mobile device in a form of patient chat data from the user-entity node 101 associated with the user 111 being monitored via a chatbot conversation agent 114 supported by the AI/ML module 107 of the MRS node 102. The patient data received from a mobile device of the patient 111 may include video, audio or textual data.

The patient chat data may have language indicator metadata representing the language of the patient 111 used during the communication. In one embodiment, the patient response data may be processed by the MRS node 102 using the pre-trained large language models (LLM). The MRS node 102 may derive the language indicator and parse out the user response data based on the language indicator metadata. In other words, the key features of the user response data may be, advantageously, derived from the user response data based on the language of the user response regardless of a form of communication.

In one embodiment, the language indicator may serve as a kind of a linguistic profile associated with the patient 111. The language indicator may guide the AI/ML module 107 in dynamically tailoring the treatment recommendation parameters for the Chabot 114 and ultimately for the patient 111. Depending on the language indicated, the MRS node 102 could engage specialized language models or apply unique natural language processing techniques optimized for that language.

The MRS node 102 may query a local patient database 103 for the historical local patient data based on the patient 111 data associated with the current patient and patient 111 sensory data associated with user entity 101 node. The MRS node 102 may acquire relevant remote patient data from a remote database 106 residing on the cloud server 105. The remote patient data in the database 106 may be collected from other third-party medical facilities. The remote patient data may be collected from the patients of the same (or similar) type, age, gender, location, language, race, etc. as the local patient 111 based on a pre-stored patient 111 profile. In one embodiment, a personalization translation on patient profile settings may be applied. For example, if a febrile seizure is at risk at a body temperature of 38.5 degrees (Celsius), but the learned (or preset) temperature for that user is 38, the alert will be triggered at 38 degrees.

The MRS node 102 may generate a feature vector or classifier data based on the patient data and sensory data and the collected heuristics data (i.e., pre-stored local patient data 103 and remote patient data 106). The MRS node 102 may ingest the feature vector/classifier data into an AI/ML module 107. The AI/ML module 107 may generate a medical recommendation predictive model(s) 108 based on the feature vector/classifier data to predict medical recommendation parameters for automatically generating medical recommendations for the patient 111 being monitored. The medical recommendation parameters may be further analyzed by the MRS node 102 prior to generation of the actual recommendation or updating the latest recommendation based on the live sensory data. In one embodiment, the updated recommendation parameters may be used for adjustment of the treatment or emergency alerts. Once the treatment is recorded, the entire or partial treatment data may be analyzed to generate a feedback report by the AI/ML module 107 based on the outputs of the medical recommendation predictive model(s) 108.

In one embodiment, the MRS node 102 may receive the medical recommendation parameters and updated parameters from a permissioned blockchain 110 ledger 109 based on a consensus from the emergency (or medical provider) medical entity nodes 113 confirming the questions and comments to be presented by the Chatbot 114 to the patient 111 of the user entity 101. Additionally, confidential historical patient-related information and previous patients'-related data and patient responses-related parameters may also be acquired from the permissioned blockchain 110. The newly acquired patient sensory data and patient response data with corresponding predicted medical recommendation parameters may be also recorded on the ledger 109 of the blockchain 110 so it can be used as training data for the medical recommendation predictive model(s) 108.

In this implementation the MRS node 102, the cloud server 105, the emergency entity nodes 113 and the user entities(s) 101 may serve as blockchain 110 peer nodes. In one embodiment, local data from the database 103 and remote data from the database 106 may be duplicated on the blockchain ledger 109 for higher security of storage.

The AI/ML module 107 may generate a medical recommendation predictive model(s) 108 to predict medical recommendation parameters in response to the specific relevant pre-stored patient-related data acquired from the blockchain 110 ledger 109. This way, the current medical recommendation parameters may be predicted based not only on the current user entity 101-related data (i.e., patient data and sensory data), but also based on the previously collected heuristics from the current patient and other patients with the same characteristics. This way, the most optimal way of handling the preventive monitoring of the patient 111, for the most likely successful implementation of patient treatment and handling of emergency scenarios may be included into the feedback report. After the data processing and the feedback report generation is completed, the related documents may be converted into unique secure NFT assets to be recorded on the blockchain to be used for future models'training.

In one embodiment, as a second round of approval, a blockchain consensus may be achieved among the emergency (or medical provider) entities 113 in order to approve the feedback report generated by the MRS node 102.

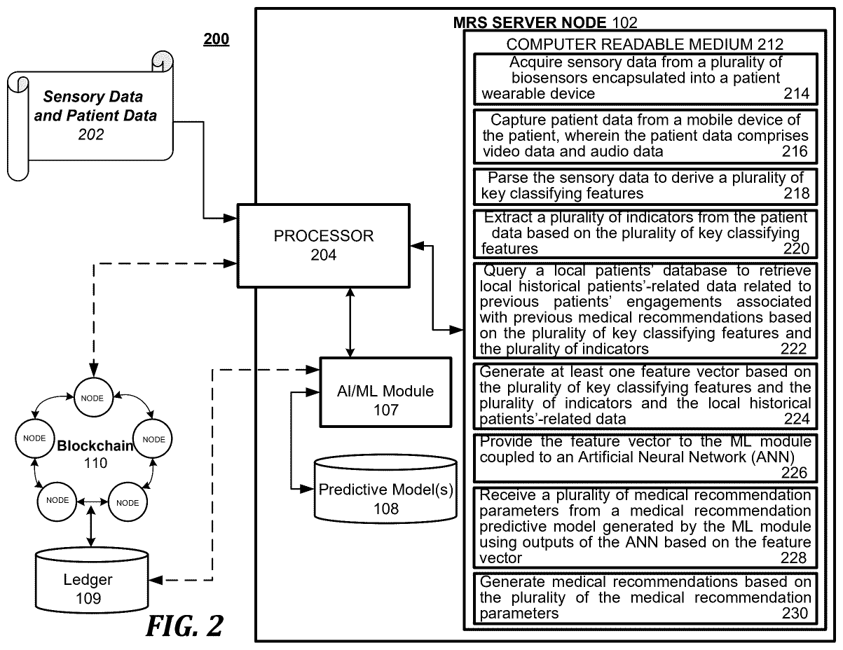

FIG. 2 illustrates a network diagram of a system including detailed features of a medical recommendations server (MRS) node consistent with the present disclosure.

Referring to FIG. 2, the example network 200 includes the MRS node 102 connected to the user entity 101 and to the emergency entity node(s) 113 (see FIGS. 1A-B) The MRS node 102 may receive the patient data and patient sensory 202. The MRS node 102 may be connected to the Chabot 114 to receive the conversation data (i.e., patient chat data) as discussed above with reference to FIGS. 1A-B.

The MRS node 102 is configured to host an AI/ML module 107. As discussed above with respect to FIGS. 1A-B, the MRS node 102 may receive the user patient data and patient sensory 202 and pre-stored patient-related data retrieved from the local and remote databases. As discussed above, the pre-stored historical patient-related data may be retrieved from the ledger 109 of the permissioned blockchain 110.

The AI/ML module 107 may generate a medical recommendation predictive model(s) 108 based on the received patient data and patient sensory 202 provided by the MRS node 102. As discussed above, the AI/ML module 107 may provide predictive outputs data in the form of medical recommendation parameters for automatic generation of the medical recommendations. The MRS node 102 may process the predictive outputs data received from the AI/ML module 107 to generate the recommendations pertaining to the hints or updated questions to be directed at patient 111 of the user entity 101 via a chatbot 114 conversation agent.

In one embodiment, the MRS node 102 may continually monitor the sensory data and may detect a parameter that deviates from a previous recorded parameter (or from a median reading value) by a margin that exceeds a threshold value pre-set for this particular parameter. For example, if patient's 111 temperature or heart rate change significantly, this may cause a change in recommendations or prompts provided to the Chabot 114. Accordingly, once the threshold is met or exceeded by at least one parameter of the patient-related data, the MRS node 102 may provide the currently acquired patient-related parameter to the AI/ML module 107 to generate an updated recommendation parameter(s) based on the current patient 111-related data.

While this example describes in detail only one MRS node 102, multiple such nodes may be connected to the network and to the blockchain 110. It should be understood that the MRS node 102 may include additional components and that some of the components described herein may be removed and/or modified without departing from a scope of the MRS node 102 disclosed herein. The MRS node 102 may be a computing device or a server computer, or the like, and may include a processor 204, which may be a semiconductor-based microprocessor, a central processing unit (CPU), an application specific integrated circuit (ASIC), a field-programmable gate array (FPGA), and/or another hardware device. Although a single processor 204 is depicted, it should be understood that the MRS node 102 may include multiple processors, multiple cores, or the like, without departing from the scope of the MRS node 102 system.

The MRS node 102 may also include a non-transitory computer readable medium 212 that may have stored thereon machine-readable instructions executable by the processor 204. Examples of the machine-readable instructions are shown as 214-230 and are further discussed below. Examples of the non-transitory computer readable medium 212 may include an electronic, magnetic, optical, or other physical storage device that contains or stores executable instructions. For example, the non-transitory computer readable medium 212 may be a Random-Access memory (RAM), an Electrically Erasable Programmable Read-Only Memory (EEPROM), a hard disk, an optical disc, or other type of storage device.

The processor 204 may fetch, decode, and execute the machine-readable instructions 214 to acquire sensory data from a plurality of biosensors encapsulated into a patient 111 wearable device (see FIG. 1A-B). The processor 204 may fetch, decode, and execute the machine-readable instructions 216 to capture patient data from a mobile device of the patient, wherein the patient data comprises video data and audio data. The processor 204 may fetch, decode, and execute the machine-readable instructions 218 to parse the sensory data to derive a plurality of key classifying features. The processor 204 may fetch, decode, and execute the machine-readable instructions 220 to extract a plurality of indicators from the patient data based on the plurality of key classifying features.

The processor 204 may fetch, decode, and execute the machine-readable instructions 222 to query a local patients'database to retrieve local historical patients'-related data related to previous patients'engagements associated with previous medical recommendations based on the plurality of key classifying features and the plurality of indicators. The processor 204 may fetch, decode, and execute the machine-readable instructions 224 to generate at least one feature vector based on the plurality of key classifying features and the plurality of indicators and the local historical patients'-related data.

The processor 204 may fetch, decode, and execute the machine-readable instructions 226 to provide the feature vector to the ML module coupled to an Artificial Neural Network (ANN). The processor 204 may fetch, decode, and execute the machine-readable instructions 228 to receive a plurality of medical recommendation parameters from a medical recommendation predictive model generated by the ML module using outputs of the ANN based on the feature vector. The processor 204 may fetch, decode, and execute the machine-readable instructions 230 to generate medical recommendations based on the plurality of the medical recommendation parameters.

As a non-limiting example, the consensual approval of the patient monitoring a treatment feedback report may be associated with a request for additional data such as proof of the treatment completion and patient recovery, etc. The permissioned blockchain 110 may be configured to use one or more smart contracts that manage transactions for multiple participating nodes and for recording the transactions on the ledger 109.

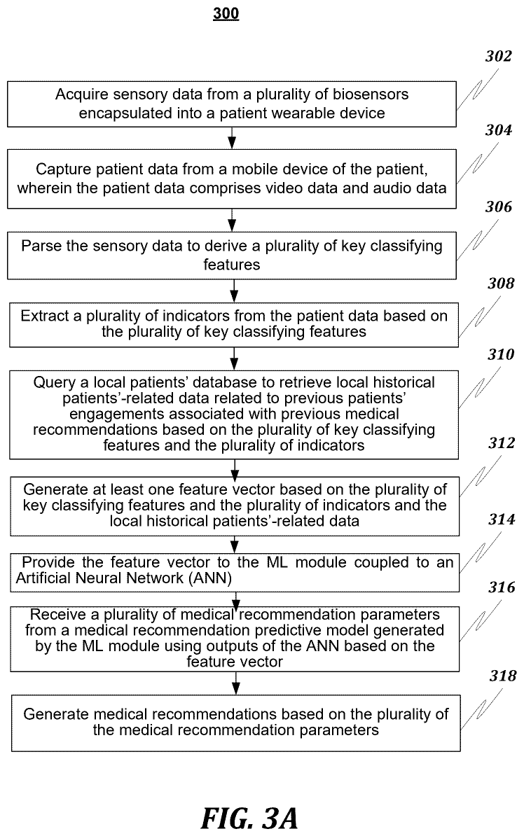

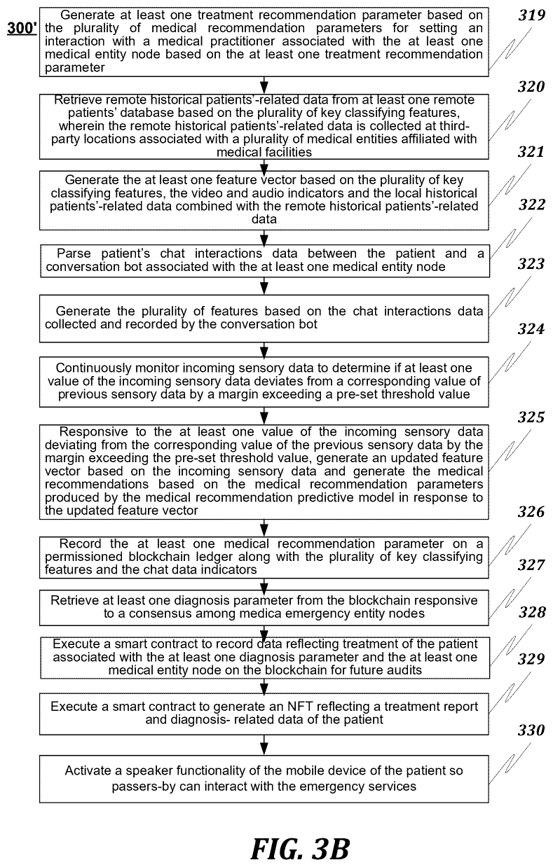

FIG. 3A illustrates a flowchart of a method for an AI-based automated real-time interactive recommendations based on predictive analytics of patient-response data and patient sensory data consistent with the present disclosure.

Referring to FIG. 3A, the method 300 may include one or more of the steps described below. FIG. 3A illustrates a flow chart of an example method executed by the MRS node 102 (see FIG. 2). It should be understood that method 300 depicted in FIG. 3A may include additional operations and that some of the operations described therein may be removed and/or modified without departing from the scope of the method 300. The description of the method 300 is also made with reference to the features depicted in FIG. 2 for purposes of illustration. Particularly, the processor 204 of the MRS node 102 may execute some or all of the operations included in the method 300.

With reference to FIG. 3A, at block 302, the processor 204 may acquire sensory data from a plurality of biosensors encapsulated into a patient wearable device. At block 304, the processor 204 may capture patient data from a mobile device of the patient, wherein the patient data comprises video data and audio data. Note that the patient data may be any of: audio data, video data, imaging data and textual data. At block 306, the processor 204 may parse the sensory data to derive a plurality of key classifying features. At block 308, the processor 204 may extract a plurality of indicators from the patient data based on the plurality of key classifying features. At block 310, the processor 204 may query a local patients'database to retrieve local historical patients'-related data related to previous patients' engagements associated with previous medical recommendations based on the plurality of key classifying features and the plurality of indicators.

At block 312, the processor 204 may generate at least one feature vector based on the plurality of key classifying features and the plurality of indicators and the local historical patients'-related data. At block 314, the processor 204 may provide the feature vector to the ML module coupled to an Artificial Neural Network (ANN). At block 316, the processor 204 may receive a plurality of medical recommendation parameters from a medical recommendation predictive model generated by the ML module using outputs of the ANN based on the feature vector. At block 318, the processor 204 may generate medical recommendations based on the plurality of the medical recommendation parameters.

FIG. 3B illustrates a further flowchart of a method for an AI-based automated real-time interactive recommendations based on predictive analytics of patient-response data and patient sensory data consistent with the present disclosure.

Referring to FIG. 3B, the method 300′ may include one or more of the steps described below. FIG. 3B illustrates a flow chart of an example method executed by the MRS node 102 (see FIG. 2). It should be understood that method 300′ depicted in FIG. 3B may include additional operations and that some of the operations described therein may be removed and/or modified without departing from the scope of the method 300′. The description of the method 300′ is also made with reference to the features depicted in FIG. 2 for purposes of illustration. Particularly, the processor 204 of the MRS 102 may execute some or all of the operations included in the method 300′.

With reference to FIG. 3B, at block 319, the processor 204 may generate at least one treatment recommendation parameter based on the plurality of medical recommendation parameters for setting an interaction with a medical practitioner associated with the at least one medical entity node based on the at least one treatment recommendation parameter.

At block 320, the processor 204 may retrieve remote historical patients'-related data from at least one remote patients'database based on the plurality of key classifying features, wherein the remote historical patients'-related data is collected at third-party locations associated with a plurality of medical entities affiliated with medical facilities. At block 321, the processor 204 may extract the plurality of classifying features based on the language identifier. At block 322, the processor 204 may generate the at least one feature vector based on the plurality of key classifying features, the video and audio indicators and the local historical patients'-related data combined with the remote historical patients'-related data. At block 323, the processor 204 may parse patient's chat interactions data between the patient and a conversation bot associated with the at least one medical entity node.

At block 324, the processor 204 may generate the plurality of features based on the chat interactions data collected and recorded by the conversation bot. At block 325, the processor 204 may continuously monitor incoming sensory data to determine if at least one value of the incoming sensory data deviates from a corresponding value of previous sensory data by a margin exceeding a pre-set threshold value.

At block 326, the processor 204 may, responsive to the at least one value of the incoming sensory data deviating from the corresponding value of the previous sensory data by the margin exceeding the pre-set threshold value, generate an updated feature vector based on the incoming sensory data and generate the medical recommendations based on the medical recommendation parameters produced by the medical recommendation predictive model in response to the updated feature vector. At block 327, the processor 204 may record the at least one medical recommendation parameter on a permissioned blockchain ledger along with the plurality of key classifying features and the chat data indicators. At block 328, the processor 204 may retrieve at least one diagnosis parameter from the blockchain responsive to a consensus among medica emergency entity nodes. At block 329, the processor 204 may execute a smart contract to record data reflecting treatment of the patient associated with the at least one diagnosis parameter and the at least one medical entity node on the blockchain for future audits. At block 330, the processor 204 may execute a smart contract to generate an NFT reflecting a treatment report and diagnosis-related data of the patient.

Further, responsive to detecting an emergency, to: alert the patient to the emergency and recommend immediate steps of dealing with the emergency; alert patient's registered care provider of the emergency, provide relevant patient-related sensory data to the care provider and offer the care provide to override the emergency; responsive to a failure of the patient or the care provider to override the emergency within a pre-set time period from the detecting of the emergency, execute a secure voice call on the mobile device of the patient to local emergency services using a conversational agent; display relevant patient data on the mobile device of the patient; locate the patient based on the sensory data and the mobile device of the patient data and relay the location of the patient to emergency services to allow emergency service personnel to reach the patient via the conversational agent; relay relevant health and sensory data of the patient to emergency services; responsive to emergency services request to act, display visual care instructions on the mobile device of the patient for a passer-by until the emergency services arrive; and terminate emergency process when the patient sensory data returns to nominal or the emergency process is overruled on the patient wearable device, the mobile device of the patient or by the patients care provider.

At block 330, the processor 204 may activate a speaker functionality of the mobile device of the patient so passers-by can interact with the emergency services.

In one disclosed embodiment, the medical recommendation predictive model may be generated by the AI/ML module 107 that may use training data sets to improve accuracy of the prediction of the medical recommendation parameters for the patient 111 (FIG. 1A-B). The medical recommendation parameters used in training data sets may be stored in a centralized local database (such as one used for storing local patient data 103 depicted in FIG. 1A). In one embodiment, a neural network may be used in the AI/ML module 107 for medical recommendation parameters modeling and feedback report generation.

In another embodiment, the AI/ML module 107 may use a decentralized storage such as a blockchain 110 (see FIG. 1B) that is a distributed storage system, which includes multiple nodes that communicate with each other. The decentralized storage includes an append-only immutable data structure resembling a distributed ledger capable of maintaining records between mutually untrusted parties. The untrusted parties are referred to herein as peers or peer nodes. Each peer maintains a copy of the parameter(s) records and no single peer can modify the records without a consensus being reached among the distributed peers. For example, the peers 101, 113, 105 and 102 (FIG. 1B) may execute a consensus protocol to validate blockchain 110 storage transactions, group the storage transactions into blocks, and build a hash chain over the blocks. This process forms the ledger 109 by ordering the storage transactions, as is necessary, for consistency. In various embodiments, a permissioned and/or a permissionless blockchain can be used. In a public or permissionless blockchain, anyone can participate without a specific identity. Public blockchains can involve assets and use consensus based on various protocols such as Proof of Work (PoW). On the other hand, a permissioned blockchain provides secure interactions among a group of entities which share a common goal such as storing recommendation parameters, but which do not fully trust one another.

This application utilizes a permissioned (private) blockchain that operates arbitrary, programmable logic, tailored to a decentralized storage scheme and referred to as “smart contracts” or “chaincodes.” In some cases, specialized chaincodes may exist for management functions and parameters which are referred to as system chaincodes. The application can further utilize smart contracts that are trusted distributed applications which leverage tamper-proof properties of the blockchain database and an underlying agreement between nodes, which is referred to as an endorsement or endorsement policy. Blockchain transactions associated with this application can be “endorsed” before being committed to the blockchain while transactions, which are not endorsed, are disregarded. An endorsement policy allows chaincodes to specify endorsers for a transaction in the form of a set of peer nodes that are necessary for endorsement. When a client sends the transaction to the peers specified in the endorsement policy, the transaction is executed to validate the transaction. After a validation, the transactions enter an ordering phase in which a consensus protocol is used to produce an ordered sequence of endorsed transactions grouped into blocks.

In the example depicted in FIG. 4, a host platform 420 (such as the MRS node 102) builds and deploys a machine learning model for predictive monitoring of assets 430. Here, the host platform 420 may be a cloud platform, an industrial server, a web server, a personal computer, a user device, and the like. Assets 430 can represent medical recommendation parameters. The blockchain 110 can be used to significantly improve both a training process 402 of the machine learning model and the medical recommendation parameters'predictive process 405 based on a trained machine learning model that uses outputs of the ANN 412. For example, in 402, rather than requiring a data scientist/engineer or other user to collect the data, historical data (heuristics—i.e., patients'-related data) may be stored by the assets 430 themselves (or through an intermediary, not shown) on the blockchain 110.

This can significantly reduce the collection time needed by the host platform 420 when performing predictive model training. For example, using smart contracts, data can be directly and reliably transferred straight from its place of origin (e.g., from the MRS node 102 or from the databases 103 and 106 depicted in FIGS. 1A-1B) to the blockchain 110. By using the blockchain 110 to ensure the security and ownership of the collected data, smart contracts may directly send the data from the assets to the entities that use the data for building a machine learning model. This allows for sharing of data among the assets 430. The collected data may be stored in the blockchain 110 based on a consensus mechanism. The consensus mechanism pulls in (permissioned nodes) to ensure that the data being recorded is verified and accurate. The data recorded is time-stamped, cryptographically signed, and immutable. It is therefore auditable, transparent, and secure.

Furthermore, training of the machine learning model on the collected data may take rounds of refinement and testing by the host platform 420. Each round may be based on additional data or data that was not previously considered to help expand the knowledge of the machine learning model. In 402, the different training and testing steps (and the data associated therewith) may be stored on the blockchain 110 by the host platform 420. Each refinement of the machine learning model (e.g., changes in variables, weights, etc.) may be stored on the blockchain 110. This, advantageously, provides verifiable proof of how the model was trained and what data was used to train the model. Furthermore, when the host platform 420 has achieved a finally trained model, the resulting model itself may be stored on the blockchain 110.

After the model has been trained, it may be deployed to a live environment where it can make recommendation-related predictions/decisions based on the execution of the final trained machine learning model using the prediction parameters. In this example, data fed back from the asset 430 may be input into the machine learning model and may be used to make event predictions such as medical recommendation parameters based on the recorded patient-related data. Determinations made by the execution of the machine learning model (e.g., approval of the treatment feedback reports, etc.) at the host platform 420 may be stored on the blockchain 110 to provide auditable/verifiable proof. As one non-limiting example, the machine learning model may predict a future change of a part of the asset 430 (the medical recommendation parameters—e.g., assessment of the sensory data). The data behind this decision may be stored by the host platform 420 on the blockchain 110.

As discussed above, in one embodiment, the features and/or the actions described and/or depicted herein can occur on or with respect to the blockchain 110. The above embodiments of the present disclosure may be implemented in hardware, in computer-readable instructions executed by a processor, in firmware, or in a combination of the above. The computer computer-readable instructions may be embodied on a computer-readable medium, such as a storage medium. For example, the computer computer-readable instructions may reside in random access memory (“RAM”), flash memory, read-only memory (“ROM”), erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

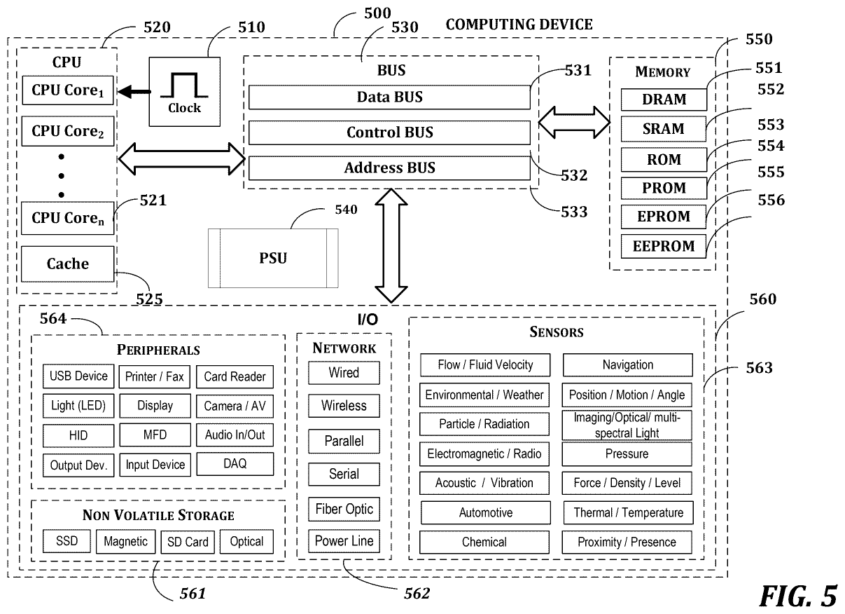

An exemplary storage medium may be coupled to the processor such that the processor may read information from, and write information to, the storage medium. In the alternative, the storage medium may be integral to the processor. The processor and the storage medium may reside in an application specific integrated circuit (“ASIC”). In the alternative embodiment, the processor and the storage medium may reside as discrete components. For example, FIG. 5 illustrates an example computing device (e.g., a server node) 500, which may represent or be integrated in any of the above-described components, etc.

FIG. 5 illustrates a block diagram of a system including computing device 500. The computing device 500 may comprise, but not be limited to the following:
Mobile computing device, such as, but is not limited to, a laptop, a tablet, a smartphone, a drone, a wearable, an embedded device, a handheld device, an Arduino, an industrial device, or a remotely operable recording device;
A supercomputer, an exa-scale supercomputer, a mainframe, or a quantum computer;
A minicomputer, wherein the minicomputer computing device comprises, but is not limited to, an IBM AS500/iSeries/System I, A DEC VAX/PDP, a HP3000, a Honeywell-Bull DPS, a Texas Instruments TI-990, or a Wang Laboratories VS Series;
A microcomputer, wherein the microcomputer computing device comprises, but is not limited to, a server, wherein a server may be rack mounted, a workstation, an industrial device, a raspberry pi, a desktop, or an embedded device;
The MRS node 102 (see FIG. 2) may be hosted on a centralized server or on a cloud computing service. Although method 300 has been described to be performed by the MRS node 102 implemented on a computing device 500, it should be understood that, in some embodiments, different operations may be performed by a plurality of the computing devices 500 in operative communication at least one network.

Embodiments of the present disclosure may comprise a computing device having a central processing unit (CPU) 520, a bus 530, a memory unit 550, a power supply unit (PSU) 550, and one or more Input/Output (I/O) units. The CPU 520 coupled to the memory unit 550 and the plurality of I/O units 560 via the bus 530, all of which are powered by the PSU 550. It should be understood that, in some embodiments, each disclosed unit may actually be a plurality of such units for the purposes of redundancy, high availability, and/or performance. The combination of the presently disclosed units is configured to perform the stages of any method disclosed herein.

Consistent with an embodiment of the disclosure, the aforementioned CPU 520, the bus 530, the memory unit 550, a PSU 550, and the plurality of I/O units 560 may be implemented in a computing device, such as computing device 500. Any suitable combination of hardware, software, or firmware may be used to implement the aforementioned units. For example, the CPU 520, the bus 530, and the memory unit 550 may be implemented with computing device 500 or any of other computing devices 500, in combination with computing device 500. The aforementioned system, device, and components are examples and other systems, devices, and components may comprise the aforementioned CPU 520, the bus 530, the memory unit 550, consistent with embodiments of the disclosure.

At least one computing device 500 may be embodied as any of the computing elements illustrated in all of the attached figures, including the MRS node 102 (FIG. 2). A computing device 500 does not need to be electronic, nor even have a CPU 520, nor bus 530, nor memory unit 550. The definition of the computing device 500 to a person having ordinary skill in the art is “A device that computes, especially a programmable [usually] electronic machine that performs high-speed mathematical or logical operations or that assembles, stores, correlates, or otherwise processes information.” Any device which processes information qualifies as a computing device 500, especially if the processing is purposeful.

With reference to FIG. 5, a system consistent with an embodiment of the disclosure may include a computing device, such as computing device 500. In a basic configuration, computing device 500 may include at least one clock module 510, at least one CPU 520, at least one bus 530, and at least one memory unit 550, at least one PSU 550, and at least one I/O 560 module, wherein I/O module may be comprised of, but not limited to a non-volatile storage sub-module 561, a communication sub-module 562, a sensors sub-module 563, and a peripherals sub-module 565.

A system consistent with an embodiment of the disclosure the computing device 500 may include the clock module 510 may be known to a person having ordinary skill in the art as a clock generator, which produces clock signals. Clock signal is a particular type of signal that oscillates between a high and a low state and is used like a metronome to coordinate actions of digital circuits. Most integrated circuits (ICs) of sufficient complexity use a clock signal in order to synchronize different parts of the circuit, cycling at a rate slower than the worst-case internal propagation delays. The preeminent example of the aforementioned integrated circuit is the CPU 520, the central component of modern computers, which relies on a clock. The only exceptions are asynchronous circuits such as asynchronous CPUs. The clock 510 can comprise a plurality of embodiments, such as, but not limited to, single-phase clock which transmits all clock signals on effectively 1 wire, two-phase clock which distributes clock signals on two wires, each with non-overlapping pulses, and four-phase clock which distributes clock signals on 5 wires.

Many computing devices 500 use a “clock multiplier” which multiplies a lower frequency external clock to the appropriate clock rate of the CPU 520. This allows the CPU 520 to operate at a much higher frequency than the rest of the computer, which affords performance gains in situations where the CPU 520 does not need to wait on an external factor (like memory 550 or input/output 560). Some embodiments of the clock 510 may include dynamic frequency change, where the time between clock edges can vary widely from one edge to the next and back again.

A system consistent with an embodiment of the disclosure the computing device 500 may include the CPU unit 520 comprising at least one CPU Core 521. A plurality of CPU cores 521 may comprise identical CPU cores 521, such as, but not limited to, homogeneous multi-core systems. It is also possible for the plurality of CPU cores 521 to comprise different CPU cores 521, such as, but not limited to, heterogeneous multi-core systems, big.LITTLE systems and some AMD accelerated processing units (APU). The CPU unit 520 reads and executes program instructions which may be used across many application domains, for example, but not limited to, general purpose computing, embedded computing, network computing, digital signal processing (DSP), and graphics processing (GPU). The CPU unit 520 may run multiple instructions on separate CPU cores 521 at the same time. The CPU unit 520 may be integrated into at least one of a single integrated circuit die and multiple dies in a single chip package. The single integrated circuit die and multiple dies in a single chip package may contain a plurality of other aspects of the computing device 500, for example, but not limited to, the clock 510, the CPU 520, the bus 530, the memory 550, and I/O 560.

The CPU unit 520 may contain cache 522 such as, but not limited to, a level 1 cache, level 2 cache, level 3 cache or combination thereof. The aforementioned cache 522 may or may not be shared amongst a plurality of CPU cores 521. The cache 522 sharing comprises at least one of message passing and inter-core communication methods may be used for the at least one CPU Core 521 to communicate with the cache 522. The inter-core communication methods may comprise, but not limited to, bus, ring, two-dimensional mesh, and crossbar. The aforementioned CPU unit 520 may employ symmetric multiprocessing (SMP) design.

The plurality of the aforementioned CPU cores 521 may comprise soft microprocessor cores on a single field programmable gate array (FPGA), such as semiconductor intellectual property cores (IP Core). The plurality of CPU cores 521 architecture may be based on at least one of, but not limited to, Complex instruction set computing (CISC), Zero instruction set computing (ZISC), and Reduced instruction set computing (RISC). At least one of the performance-enhancing methods may be employed by the plurality of the CPU cores 521, for example, but not limited to Instruction-level parallelism (ILP) such as, but not limited to, superscalar pipelining, and Thread-level parallelism (TLP).

Consistent with the embodiments of the present disclosure, the aforementioned computing device 500 may employ a communication system that transfers data between components inside the aforementioned computing device 500, and/or the plurality of computing devices 500. The aforementioned communication system will be known to a person having ordinary skill in the art as a bus 530. The bus 530 may embody internal and/or external plurality of hardware and software components, for example, but not limited to a wire, optical fiber, communication protocols, and any physical arrangement that provides the same logical function as a parallel electrical bus. The bus 530 may comprise at least one of, but not limited to a parallel bus, wherein the parallel bus carry data words in parallel on multiple wires, and a serial bus, wherein the serial bus carry data in bit-serial form. The bus 530 may embody a plurality of topologies, for example, but not limited to, a multidrop/electrical parallel topology, a daisy chain topology, and a connected by switched hubs, such as USB bus. The bus 530 may comprise a plurality of embodiments, for example, but not limited to:
Internal data bus (data bus) 531/Memory bus
Control bus 532
Address bus 533
System Management Bus (SMBus)
Front-Side-Bus (FSB)
External Bus Interface (EBI)
Local bus
Expansion bus
Lightning bus
Controller Area Network (CAN bus)
Camera Link
ExpressCard
Advanced Technology management Attachment (ATA), including embodiments and derivatives such as, but not limited to, Integrated Drive Electronics (IDE)/Enhanced IDE (EIDE), ATA Packet Interface (ATAPI), Ultra-Direct Memory Access (UDMA), Ultra ATA (UATA)/Parallel ATA (PATA)/Serial ATA (SATA), CompactFlash (CF) interface, Consumer Electronics ATA (CE-ATA)/Fiber Attached Technology Adapted (FATA), Advanced Host Controller Interface (AHCI), SATA Express (SATAe)/External SATA (eSATA), including the powered embodiment eSATAp/Mini-SATA (mSATA), and Next Generation Form Factor (NGFF)/M.2.
Small Computer System Interface (SCSI)/Serial Attached SCSI (SAS)
HyperTransport
InfiniBand
RapidIO
Mobile Industry Processor Interface (MIPI)
Coherent Processor Interface (CAPI)
Plug-n-play
1-Wire
Peripheral Component Interconnect (PCI), including embodiments such as, but not limited to, Accelerated Graphics Port (AGP), Peripheral Component Interconnect eXtended (PCI-X), Peripheral Component Interconnect Express (PCI-e) (e.g., PCI Express Mini Card, PCI Express M.2 [Mini PCIe v2], PCI Express External Cabling [ePCIe], and PCI Express OCuLink [Optical Copper{Cu} Link]), Express Card, AdvancedTCA, AMC, Universal IO, Thunderbolt/Mini DisplayPort, Mobile PCIe (M-PCIe), U.2, and Non-Volatile Memory Express (NVMe)/Non-Volatile Memory Host Controller Interface Specification (NVMHCIS).
Industry Standard Architecture (ISA), including embodiments such as, but not limited to Extended ISA (EISA), PC/XT-bus/PC/AT-bus/PC/105bus (e.g., PC/105-Plus, PCI/105-Express, PCI/105, and PCI-105), and Low Pin Count (LPC).
Music Instrument Digital Interface (MIDI)
Universal Serial Bus (USB), including embodiments such as, but not limited to, Media Transfer Protocol (MTP)/Mobile High-Definition Link (MHL), Device Firmware Upgrade (DFU), wireless USB, InterChip USB, IEEE 1395 Interface/Firewire, Thunderbolt, and eXtensible Host Controller Interface (xHCI).

Consistent with the embodiments of the present disclosure, the aforementioned computing device 500 may employ hardware integrated circuits that store information for immediate use in the computing device 500, known to the person having ordinary skill in the art as primary storage or memory 550. The memory 550 operates at high speed, distinguishing it from the non-volatile storage sub-module 561, which may be referred to as secondary or tertiary storage, which provides slow-to-access information but offers higher capacities at lower cost. The contents contained in memory 550, may be transferred to secondary storage via techniques such as, but not limited to, virtual memory and swap. The memory 550 may be associated with addressable semiconductor memory, such as integrated circuits consisting of silicon-based transistors, used for example as primary storage but also other purposes in the computing device 500. The memory 550 may comprise a plurality of embodiments, such as, but not limited to volatile memory, non-volatile memory, and semi-volatile memory. It should be understood by a person having ordinary skill in the art that the ensuing are non-limiting examples of the aforementioned memory:
Volatile memory which requires power to maintain stored information, for example, but not limited to, Dynamic Random-Access Memory (DRAM) 551, Static Random-Access Memory (SRAM) 552, CPU Cache memory 525, Advanced Random-Access Memory (A-RAM), and other types of primary storage such as Random-Access Memory (RAM).
Non-volatile memory which can retain stored information even after power is removed, for example, but not limited to, Read-Only Memory (ROM) 553, Programmable ROM (PROM) 555, Erasable PROM (EPROM) 555, Electrically Erasable PROM (EEPROM) 556 (e.g., flash memory and Electrically Alterable PROM [EAPROM]), Mask ROM (MROM), One Time Programmable (OTP) ROM/Write Once Read Many (WORM), Ferroelectric RAM (FeRAM), Parallel Random-Access Machine (PRAM), Split-Transfer Torque RAM (STT-RAM), Silicon Oxime Nitride Oxide Silicon (SONOS), Resistive RAM (RRAM), Nano RAM (NRAM), 3D XPoint, Domain-Wall Memory (DWM), and millipede memory.
Semi-volatile memory which may have some limited non-volatile duration after power is removed but loses data after said duration has passed. Semi-volatile memory provides high performance, durability, and other valuable characteristics typically associated with volatile memory, while providing some benefits of true non-volatile memory. The semi-volatile memory may comprise volatile and non-volatile memory and/or volatile memory with battery to provide power after power is removed. The semi-volatile memory may comprise, but not limited to spin-transfer torque RAM (STT-RAM).
Consistent with the embodiments of the present disclosure, the aforementioned computing device 500 may employ the communication system between an information processing system, such as the computing device 500, and the outside world, for example, but not limited to, human, environment, and another computing device 500. The aforementioned communication system will be known to a person having ordinary skill in the art as I/O 560. The I/O module 560 regulates a plurality of inputs and outputs with regard to the computing device 500, wherein the inputs are a plurality of signals and data received by the computing device 500, and the outputs are the plurality of signals and data sent from the computing device 500. The I/O module 560 interfaces a plurality of hardware, such as, but not limited to, non-volatile storage 561, communication devices 562, sensors 563, and peripherals 565. The plurality of hardware is used by at least one of, but not limited to, human, environment, and another computing device 500 to communicate with the present computing device 500. The I/O module 560 may comprise a plurality of forms, for example, but not limited to channel I/O, port mapped I/O, asynchronous I/O, and Direct Memory Access (DMA).
Consistent with the embodiments of the present disclosure, the aforementioned computing device 500 may employ the non-volatile storage sub-module 561, which may be referred to by a person having ordinary skill in the art as one of secondary storage, external memory, tertiary storage, off-line storage, and auxiliary storage. The non-volatile storage sub-module 561 may not be accessed directly by the CPU 520 without using an intermediate area in the memory 550. The non-volatile storage sub-module 561 does not lose data when power is removed and may be two orders of magnitude less costly than storage used in memory modules, at the expense of speed and latency. The non-volatile storage sub-module 561 may comprise a plurality of forms, such as, but not limited to, Direct Attached Storage (DAS), Network Attached Storage (NAS), Storage Area Network (SAN), nearline storage, Massive Array of Idle Disks (MAID), Redundant Array of Independent Disks (RAID), device mirroring, off-line storage, and robotic storage. The non-volatile storage sub-module (561) may comprise a plurality of embodiments, such as, but not limited to:
Optical storage, for example, but not limited to, Compact Disk (CD) (CD-ROM/CD-R/CD-RW), Digital Versatile Disk (DVD) (DVD-ROM/DVD-R/DVD+R/DVD-RW/DVD+RW/DVD±RW/DVD+R DL/DVD-RAM/HD-DVD), Blu-ray Disk (BD) (BD-ROM/BD-R/BD-RE/BD-R DL/BD-RE DL), and Ultra-Density Optical (UDO).
Semiconductor storage, for example, but not limited to, flash memory, such as, but not limited to, USB flash drive, Memory card, Subscriber Identity Module (SIM) card, Secure Digital (SD) card, Smart Card, CompactFlash (CF) card, Solid-State Drive (SSD) and memristor.
Magnetic storage such as, but not limited to, Hard Disk Drive (HDD), tape drive, carousel memory, and Card Random-Access Memory (CRAM).
Phase-change memory
Holographic data storage such as Holographic Versatile Disk (HVD).
Molecular Memory
Deoxyribonucleic Acid (DNA) digital data storage

Consistent with the embodiments of the present disclosure, the aforementioned computing device 500 may employ the communication sub-module 562 as a subset of the I/O 560, which may be referred to by a person having ordinary skill in the art as at least one of, but not limited to, computer network, data network, and network. The network allows computing devices 500 to exchange data using connections, which may be known to a person having ordinary skill in the art as data links, between network nodes. The nodes comprise network computer devices 500 that originate, route, and terminate data. The nodes are identified by network addresses and can include a plurality of hosts consistent with the embodiments of a computing device 500. The aforementioned embodiments include, but not limited to personal computers, phones, servers, drones, and networking devices such as, but not limited to, hubs, switches, routers, modems, and firewalls.

Two nodes can be networked together, when one computing device 500 is able to exchange information with the other computing device 500, whether or not they have a direct connection with each other. The communication sub-module 562 supports a plurality of applications and services, such as, but not limited to World Wide Web (WWW), digital video and audio, shared use of application and storage computing devices 500, printers/scanners/fax machines, email/online chat/instant messaging, remote control, distributed computing, etc. The network may comprise a plurality of transmission mediums, such as, but not limited to conductive wire, fiber optics, and wireless. The network may comprise a plurality of communications protocols to organize network traffic, wherein application-specific communications protocols are layered, may be known to a person having ordinary skill in the art as carried as payload, over other more general communications protocols. The plurality of communications protocols may comprise, but not limited to, IEEE 802, ethernet, Wireless LAN (WLAN/Wi-Fi), Internet Protocol (IP) suite (e.g., TCP/IP, UDP, Internet Protocol version 5 [IPv5], and Internet Protocol version 6 [IPv6]), Synchronous Optical Networking (SONET)/Synchronous Digital Hierarchy (SDH), Asynchronous Transfer Mode (ATM), and cellular standards (e.g., Global System for Mobile Communications [GSM], General Packet Radio Service [GPRS], Code-Division Multiple Access [CDMA], and Integrated Digital Enhanced Network [IDEN]).

The communication sub-module 562 may comprise a plurality of size, topology, traffic control mechanism and organizational intent. The communication sub-module 562 may comprise a plurality of embodiments, such as, but not limited to:
Wired communications, such as, but not limited to, coaxial cable, phone lines, twisted pair cables (ethernet), and InfiniBand.
Wireless communications, such as, but not limited to, communications satellites, cellular systems, radio frequency/spread spectrum technologies, IEEE 802.11 Wi-Fi, Bluetooth, NFC, free-space optical communications, terrestrial microwave, and Infrared (IR) communications. Cellular systems embody technologies such as, but not limited to, 3G, 5G (such as WiMax and LTE), and 5G (short and long wavelength).
Parallel communications, such as, but not limited to, LPT ports.
Serial communications, such as, but not limited to, RS-232 and USB.
Fiber Optic communications, such as, but not limited to, Single-mode optical fiber (SMF) and Multi-mode optical fiber (MMF).
Power Line and wireless communications

The aforementioned network may comprise a plurality of layouts, such as, but not limited to, bus network such as ethernet, star network such as Wi-Fi, ring network, mesh network, fully connected network, and tree network. The network can be characterized by its physical capacity or its organizational purpose. Use of the network, including user authorization and access rights, differ accordingly. The characterization may include, but not limited to nanoscale network, Personal Area Network (PAN), Local Area Network (LAN), Home Area Network (HAN), Storage Area Network (SAN), Campus Area Network (CAN), backbone network, Metropolitan Area Network (MAN), Wide Area Network (WAN), enterprise private network, Virtual Private Network (VPN), and Global Area Network (GAN).

Consistent with the embodiments of the present disclosure, the aforementioned computing device 500 may employ the sensors sub-module 563 as a subset of the I/O 560. The sensors sub-module 563 comprises at least one of the devices, modules, and subsystems whose purpose is to detect events or changes in its environment and send the information to the computing device 500. Sensors are sensitive to the measured property, are not sensitive to any property not measured, but may be encountered in its application, and do not significantly influence the measured property. The sensors sub-module 563 may comprise a plurality of digital devices and analog devices, wherein if an analog device is used, an Analog to Digital (A-to-D) converter must be employed to interface the said device with the computing device 500. The sensors may be subject to a plurality of deviations that limit sensor accuracy. The sensors sub-module 563 may comprise a plurality of embodiments, such as, but not limited to, chemical sensors, automotive sensors, acoustic/sound/vibration sensors, electric current/electric potential/magnetic/radio sensors, environmental/weather/moisture/humidity sensors, flow/fluid velocity sensors, ionizing radiation/particle sensors, navigation sensors, position/angle/displacement/distance/speed/acceleration sensors, imaging/optical/light sensors, pressure sensors, force/density/level sensors, thermal/temperature sensors, and proximity/presence sensors. It should be understood by a person having ordinary skill in the art that the ensuing are non-limiting examples of the aforementioned sensors:

Chemical sensors, such as, but not limited to, breathalyzer, carbon dioxide sensor, carbon monoxide/smoke detector, catalytic bead sensor, chemical field-effect transistor, chemiresistor, electrochemical gas sensor, electronic nose, electrolyte-insulator-semiconductor sensor, energy-dispersive X-ray spectroscopy, fluorescent chloride sensors, holographic sensor, hydrocarbon dew point analyzer, hydrogen sensor, hydrogen sulfide sensor, infrared point sensor, ion-selective electrode, nondispersive infrared sensor, microwave chemistry sensor, nitrogen oxide sensor, olfactometer, optode, oxygen sensor, ozone monitor, pellistor, pH glass electrode, potentiometric sensor, redox electrode, zinc oxide nanorod sensor, and biosensors (such as nano-sensors).

Automotive sensors, such as, but not limited to, air flow meter/mass airflow sensor, air-fuel ratio meter, AFR sensor, blind spot monitor, engine coolant/exhaust gas/cylinder head/transmission fluid temperature sensor, hall effect sensor, wheel/automatic transmission/turbine/vehicle speed sensor, airbag sensors, brake fluid/engine crankcase/fuel/oil/tire pressure sensor, camshaft/crankshaft/throttle position sensor, fuel/oil level sensor, knock sensor, light sensor, MAP sensor, oxygen sensor (o2), parking sensor, radar sensor, torque sensor, variable reluctance sensor, and water-in-fuel sensor.
Acoustic, sound and vibration sensors, such as, but not limited to, microphone, lace sensor (guitar pickup), seismometer, sound locator, geophone, and hydrophone.
Electric current, electric potential, magnetic, and radio sensors, such as, but not limited to, current sensor, Daly detector, electroscope, electron multiplier, faraday cup, galvanometer, hall effect sensor, hall probe, magnetic anomaly detector, magnetometer, magnetoresistance, MEMS magnetic field sensor, metal detector, planar hall sensor, radio direction finder, and voltage detector.
Environmental, weather, moisture, and humidity sensors, such as, but not limited to, actinometer, air pollution sensor, bedwetting alarm, ceilometer, dew warning, electrochemical gas sensor, fish counter, frequency domain sensor, gas detector, hook gauge evaporimeter, humistor, hygrometer, leaf sensor, lysimeter, pyranometer, pyrgeometer, psychrometer, rain gauge, rain sensor, seismometers, SNOTEL, snow gauge, soil moisture sensor, stream gauge, and tide gauge.
Flow and fluid velocity sensors, such as, but not limited to, air flow meter, anemometer, flow sensor, gas meter, mass flow sensor, and water meter.
Ionizing radiation and particle sensors, such as, but not limited to, cloud chamber, Geiger counter, Geiger-Muller tube, ionization chamber, neutron detection, proportional counter, scintillation counter, semiconductor detector, and thermos-luminescent dosimeter.
Navigation sensors, such as, but not limited to, air speed indicator, altimeter, attitude indicator, depth gauge, fluxgate compass, gyroscope, inertial navigation system, inertial reference unit, magnetic compass, MHD sensor, ring laser gyroscope, turn coordinator, variometer, vibrating structure gyroscope, and yaw rate sensor.
Position, angle, displacement, distance, speed, and acceleration sensors, such as, but not limited to, accelerometer, displacement sensor, flex sensor, free fall sensor, gravimeter, impact sensor, laser rangefinder, LIDAR, odometer, photoelectric sensor, position sensor such as, but not limited to, GPS or Glonass, angular rate sensor, shock detector, ultrasonic sensor, tilt sensor, tachometer, ultra-wideband radar, variable reluctance sensor, and velocity receiver.
Imaging, optical and light sensors, such as, but not limited to, CMOS sensor, LiDAR, multi-spectral light sensor, colorimeter, contact image sensor, electro-optical sensor, infra-red sensor, kinetic inductance detector, LED as light sensor, light-addressable potentiometric sensor, Nichols radiometer, fiber-optic sensors, optical position sensor, thermopile laser sensor, photodetector, photodiode, photomultiplier tubes, phototransistor, photoelectric sensor, photoionization detector, photomultiplier, photoresistor, photo-switch, phototube, scintillometer, Shack-Hartmann, single-photon avalanche diode, superconducting nanowire single-photon detector, transition edge sensor, visible light photon counter, and wavefront sensor.
Pressure sensors, such as, but not limited to, barograph, barometer, boost gauge, bourdon gauge, hot filament ionization gauge, ionization gauge, McLeod gauge, Oscillating U-tube, permanent downhole gauge, piezometer, Pirani gauge, pressure sensor, pressure gauge, tactile sensor, and time pressure gauge.
Force, Density, and Level sensors, such as, but not limited to, bhangmeter, hydrometer, force gauge or force sensor, level sensor, load cell, magnetic level or nuclear density sensor or strain gauge, piezo capacitive pressure sensor, piezoelectric sensor, torque sensor, and viscometer.
Thermal and temperature sensors, such as, but not limited to, bolometer, bimetallic strip, calorimeter, exhaust gas temperature gauge, flame detection/pyrometer, Gardon gauge, Golay cell, heat flux sensor, microbolometer, microwave radiometer, net radiometer, infrared/quartz/resistance thermometer, silicon bandgap temperature sensor, thermistor, and thermocouple.
Proximity and presence sensors, such as, but not limited to, alarm sensor, doppler radar, motion detector, occupancy sensor, proximity sensor, passive infrared sensor, reed switch, stud finder, triangulation sensor, touch switch, and wired glove.

Consistent with the embodiments of the present disclosure, the aforementioned computing device 500 may employ the peripherals sub-module 562 as a subset of the I/O 560. The peripheral sub-module 565 comprises ancillary devices used to put information into and get information out of the computing device 500. There are 3 categories of devices comprising the peripheral sub-module 565, which exist based on their relationship with the computing device 500, input devices, output devices, and input/output devices. Input devices send at least one of data and instructions to the computing device 500. Input devices can be categorized based on, but not limited to:
Modality of input, such as, but not limited to, mechanical motion, audio, visual, and tactile.
Whether the input is discrete, such as but not limited to, pressing a key, or continuous such as, but not limited to position of a mouse.
The number of degrees of freedom involved, such as, but not limited to, two-dimensional mice vs three-dimensional mice used for Computer-Aided Design (CAD) applications.

Output devices provide output from the computing device 500. Output devices convert electronically generated information into a form that can be presented to humans. Input /utput devices that perform both input and output functions. It should be understood by a person having ordinary skill in the art that the ensuing are non-limiting embodiments of the aforementioned peripheral sub-module 565:

### Input Devices

Human Interface Devices (HID), such as, but not limited to, pointing device (e.g., mouse, touchpad, joystick, touchscreen, game controller/gamepad, remote, light pen, light gun, Wii remote, jog dial, shuttle, and knob), keyboard, graphics tablet, digital pen, gesture recognition devices, magnetic ink character recognition, Sip-and-Puff (SNP) device, and Language Acquisition Device (LAD).
High degree of freedom devices, that require up to six degrees of freedom such as, but not limited to, camera gimbals, Cave Automatic Virtual Environment (CAVE), and virtual reality systems.
Video Input devices are used to digitize images or video from the outside world into the computing device 500. The information can be stored in a multitude of formats depending on the user's requirement. Examples of types of video input devices include, but not limited to, digital camera, digital camcorder, portable media player, webcam, Microsoft Kinect, image scanner, fingerprint scanner, barcode reader, 3D scanner, laser rangefinder, eye gaze tracker, computed tomography, magnetic resonance imaging, positron emission tomography, medical ultrasonography, TV tuner, and iris scanner.
Audio input devices are used to capture sound. In some cases, an audio output device can be used as an input device, in order to capture produced sound. Audio input devices allow a user to send audio signals to the computing device 500 for at least one of processing, recording, and carrying out commands. Devices such as microphones allow users to speak to the computer in order to record a voice message or navigate software. Aside from recording, audio input devices are also used with speech recognition software. Examples of types of audio input devices include, but not limited to microphone, Musical Instrument Digital Interface (MIDI) devices such as, but not limited to a keyboard, and headset.
Data Acquisition (DAQ) devices convert at least one of analog signals and physical parameters to digital values for processing by the computing device 500. Examples of DAQ devices may include, but not limited to, Analog to Digital Converter (ADC), data logger, signal conditioning circuitry, multiplexer, and Time to Digital Converter (TDC).

Output Devices may further comprise, but not be limited to:
Display devices, which convert electrical information into visual form, such as, but not limited to, monitor, TV, projector, and Computer Output Microfilm (COM). Display devices can use a plurality of underlying technologies, such as, but not limited to, Cathode-Ray Tube (CRT), Thin-Film Transistor (TFT), Liquid Crystal Display (LCD), Organic Light-Emitting Diode (OLED), MicroLED, E Ink Display (ePaper) and Refreshable Braille Display (Braille Terminal).

Printers, such as, but not limited to, inkjet printers, laser printers, 3D printers, solid ink printers and plotters.
Audio and Video (AV) devices, such as, but not limited to, speakers, headphones, amplifiers and lights, which include lamps, strobes, DJ lighting, stage lighting, architectural lighting, special effect lighting, and lasers.
Other devices such as Digital to Analog Converter (DAC)

Input/Output Devices may further comprise, but not be limited to, touchscreens, networking device (e.g., devices disclosed in network 562 sub-module), data storage device (non-volatile storage 561), facsimile (FAX), and graphics/sound cards.

All rights including copyrights in the code included herein are vested in and the property of the Applicant. The Applicant retains and reserves all rights in the code included herein, and grants permission to reproduce the material only in connection with reproduction of the granted patent and for no other purpose.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

The following is claimed:

1. A system for generation of medical recommendation based on patient-related data, comprising:
a processor of a medical recommendations server (MRS) node configured to host a machine learning (ML) module and connected to a patient entity node and to at least one medical emergency entity node over a network; and
a memory on which are stored machine-readable instructions that when executed by the processor, cause the processor to:
acquire sensory data from a plurality of biosensors encapsulated into a patient wearable device;
acquire patient data from a mobile device of the patient, wherein the patient data comprises video data and audio data;
parse the sensory data to derive a plurality of key classifying features;
extract a plurality of indicators from the patient data based on the plurality of key classifying features;
query a local patients'database to retrieve local historical patients'-related data related to previous patients'engagements associated with previous medical recommendations based on the plurality of key classifying features and the plurality of indicators;
generate at least one feature vector based on the plurality of key classifying features and the plurality of indicators and the local historical patients'-related data; and
provide the feature vector to the ML module coupled to an Artificial Neural Network (ANN);
receive a plurality of medical recommendation parameters from a medical recommendation predictive model generated by the ML module using outputs of the ANN based on the feature vector; and
generate medical recommendations based on the plurality of the medical recommendation parameters.

2. The system of claim 1, wherein the instructions further cause the processor to generate at least one treatment recommendation parameter based on the plurality of medical recommendation parameters for setting an interaction with a medical practitioner associated with the at least one medical entity node based on the at least one treatment recommendation parameter.

3. The system of claim 1, wherein the instructions further cause the processor to retrieve remote historical patients'-related data from at least one remote patients'database based on the plurality of key classifying features, wherein the remote historical patients'-related data is collected at third-party locations associated with a plurality of medical entities affiliated with medical facilities.

4. The system of claim 3, wherein the instructions further cause the processor to generate the at least one feature vector based on the plurality of key classifying features, the video and audio indicators and the local historical patients'-related data combined with the remote historical patients'-related data.

5. The system of claim 1, wherein the instructions further cause the processor to parse patient's chat interactions data between the patient and a conversation bot associated with the at least one medical entity node.

6. The system of claim 5, wherein the instructions further cause the processor to generate the plurality of features based on the chat interactions data collected and recorded by the conversation bot.

7. The system of claim 1, wherein the instructions further cause the processor to continuously monitor incoming sensory data to determine if at least one value of the incoming sensory data deviates from a corresponding value of previous sensory data by a margin exceeding a pre-set threshold value.

8. The system of claim 7, wherein the instructions further cause the processor to, responsive to the at least one value of the incoming sensory data deviating from the corresponding value of the previous sensory data by the margin exceeding the pre-set threshold value, generate an updated feature vector based on the incoming sensory data and generate the medical recommendations based on the medical recommendation parameters produced by the medical recommendation predictive model in response to the updated feature vector.

9. The system of claim 1, wherein the instructions further cause the processor to record the at least one medical recommendation parameter on a permissioned blockchain ledger along with the plurality of key classifying features and the chat data indicators.

10. The system of claim 9, wherein the instructions further cause the processor to retrieve at least one diagnosis parameter from the blockchain responsive to a consensus among medica emergency entity nodes.

11. The system of claim 10, wherein the instructions further cause the processor to execute a smart contract to record data reflecting treatment of the patient associated with the at least one diagnosis parameter and the at least one medical entity node on the blockchain for future audits.

12. The system of claim 10, wherein the instructions further cause the processor to execute a smart contract to generate an NFT reflecting a treatment report and diagnosis-related data of the patient.

13. The system of claim 1, wherein the instructions further cause the processor, responsive to detecting an emergency, to:
alert the patient to the emergency and recommend immediate steps of dealing with the emergency;
alert patient's registered care provider of the emergency, provide relevant patient-related sensory data to the care provider and offer the care provide to override the emergency;
responsive to a failure of the patient or the care provider to override the emergency within a pre-set time period from the detecting of the emergency, execute a secure voice call on the mobile device of the patient to local emergency services using a conversational agent;
display relevant patient data on the mobile device of the patient;
locate the patient based on the sensory data and the mobile device of the patient data and relay the location of the patient to emergency services to allow emergency service personnel to reach the patient via the conversational agent;
relay relevant health and sensory data of the patient to emergency services;
responsive to emergency services request to act, display visual care instructions on the mobile device of the patient for a passer-by until the emergency services arrive; and
terminate emergency process when the patient sensory data returns to nominal or the emergency process is overruled on the patient wearable device, the mobile device of the patient or by the patients care provider.

14. The system of claim 13, wherein the instructions further cause the processor to activate a speaker functionality of the mobile device of the patient so passers-by can interact with the emergency services.

15. A method for generation of medical recommendation based on patient-related data, comprising:
acquiring, by a medical recommendations server (MRS) node, sensory data from a plurality of biosensors encapsulated into a patient wearable device;
capturing, by the MRS node, patient data from a mobile device of the patient, wherein the patient data comprises video data and audio data;
parsing, by the MRS node, the sensory data to derive a plurality of key classifying features;
extracting, by the MRS node, a plurality of indicators from the patient data based on the plurality of key classifying features;
querying, by the MRS node, a local patients'database to retrieve local historical patients'-related data related to previous patients'engagements associated with previous medical recommendations based on the plurality of key classifying features and the plurality of indicators;
generating, by the MRS node, at least one feature vector based on the plurality of key classifying features and the plurality of indicators and the local historical patients'-related data; and
providing, by the MRS node, the feature vector to a ML module coupled to an Artificial Neural Network (ANN);
receiving, by the MRS node, a plurality of medical recommendation parameters from a medical recommendation predictive model generated by the ML module using outputs of the ANN based on the feature vector; and
generating, by the MRS node, medical recommendations based on the plurality of the medical recommendation parameters.

16. The method of claim 15, further comprising retrieving remote historical patients'-related data from at least one remote patients'database based on the plurality of key classifying features, wherein the remote historical patients'-related data is collected at third-party locations associated with a plurality of medical entities affiliated with medical facilities.

17. The method of claim 16, further comprising generating the at least one feature vector based on the plurality of key classifying features, the video and audio indicators and the local historical patients'-related data combined with the remote historical patients'-related data.

18. The method of claim 15, further comprising continuously monitoring incoming sensory data to determine if at least one value of the incoming sensory data deviates from a corresponding value of previous sensory data by a margin exceeding a pre-set threshold value.

19. The method of claim 18, further comprising, responsive to the at least one value of the incoming sensory data deviating from the corresponding value of the previous sensory data by the margin exceeding the pre-set threshold value, generating an updated feature vector based on the incoming sensory data and generating the medical recommendations based on the medical recommendation parameters produced by the medical recommendation predictive model in response to the updated feature vector.

20. A non-transitory computer-readable medium comprising instructions, that when read by a processor, cause the processor to perform:
acquiring sensory data from a plurality of biosensors encapsulated into a patient wearable device;
capturing patient data from a mobile device of the patient, wherein the patient data comprises video data and audio data;
parsing the sensory data to derive a plurality of key classifying features;
extracting a plurality of indicators from the patient data based on the plurality of key classifying features;
querying a local patients'database to retrieve local historical patients'-related data related to previous patients'engagements associated with previous medical recommendations based on the plurality of key classifying features and the plurality of indicators;
generating at least one feature vector based on the plurality of key classifying features and the plurality of indicators and the local historical patients'-related data; and
providing the feature vector to a ML module coupled to an Artificial Neural Network (ANN);
receiving a plurality of medical recommendation parameters from a medical recommendation predictive model generated by the ML module using outputs of the ANN based on the feature vector; and
generating medical recommendations based on the plurality of the medical recommendation parameters.
